(19) United States
(12) Patent Application Publication
(10) Pub. No.: US 2013/0102955 A1
(43) Pub. Date: Apr. 25, 2013

(54) APPARATUS AND METHOD FOR PERFORMING PHACOEMULSIFICATION

(71) Applicant: Richard S. KOPLIN, New York, NY (US)

(72) Inventor: Richard S. KOPLIN, New York, NY (US)

(21) Appl. No.: 13/625,158

(22) Filed: Sep. 24, 2012

Related U.S. Application Data
(60) Provisional application No. 61/570,555, filed on Dec. 14, 2011.
(60) Provisional application No. 61/539,016, filed on Sep. 26, 2011.

(51) Int. Cl. A61F 9/007

(52) U.S. Cl. CPC A61F 9/00745
USPC 604/22

(57) ABSTRACT

An apparatus provides mechanical energy to vibrate a tip. The tip preferably formed of multiple prongs positioned approximately circumferentially (either symmetrically or asymmetrically) around an orifice. The tip is designed to emulsify a cataractous lens and to collect the resulting detritus through an aspiration aperture. An irrigating aperture is provided at a point spaced away from the tip and forms a plume or river of irrigating fluid that less directly confronts lens detritus at the needle tip and therefore collects this detritus in a more efficient and salutary manner as it flows to the aspiration aperture, said plume extend around the prongs. The prongs can be driven at either subsonic frequencies or ultrasonic frequencies.

### RELATED APPLICATIONS

This application claims priority to U.S. Provisional applications Ser. No. 61/539,016 filed Sep. 26, 2011; and Ser. No. 61/570,555 filed Dec. 14, 2011, both incorporated herein by reference in their entirety.

### BACKGROUND OF THE INVENTION

A. Field of Invention

This invention pertains to an apparatus for performing phacoemulsification and fluid infusion and maintenance within the eye. The apparatus includes a sleeve with lateral outlets for ejecting fluid into the eye in a predetermined pattern selected to prevent detritus resulting from the phacoemulsification to migrate away from the site and, possibly into the eye. The apparatus may also include a needle having several circumferential slits directing sonic waves at the site of interest.

B. Description of the Prior Art

Phacoemulsification is a procedure used to break up and remove the natural lens from the capsular bag within the eye of a person. Most often the procedure is used as a means of treating a person having cataracts. The procedure involves making a small incision in the eye and introducing a thin needle formed on a horn through the incision. The horn is coupled to an ultrasonic generator that vibrates the needle in a predetermined ultrasonic frequency range causing the natural lens to fragment and become emulsate. The nuclear emulsate within capsular bag is aspirated during this process and simultaneously irrigation (infusion) produces a stabilizing effect in the anterior and posterior chambers, keeping the eye inflated.

To complete the operation an intraocular lens implant is then inserted into the capsular bag (usually through the same incision incorporating the ultrasonic hand-piece).

While the technology has for the most part been broadly accepted as the community norm presently available equipment is noted to have several disadvantages. Of these disadvantages is that in a typical equipment for performing phacoemulsification, the infusion and aspiration functions (via the central bore of the phacoemulsification needle) are inherently in close proximity. Under certain conditions the infusion fluid stream within the eye may interact in a deleterious manner tending to drive lens detritus away from the aspiration flow. Because of this phenomenon the phacoemulsification process is not only inherently less efficient but nuclear or other lens material may be driven far afield of the hand-piece, and become lost to the surgical field, and at times remain in the eye in various hidden anatomical locations.

Another disadvantage of the existing apparatus is that the ultrasonic generator and the needle being vibrated has a tendency to generate excessive heat and must be cooled by infusion fluid to insure that the heat thus generated does not cause any internal injuries in the eye. A further disadvantage of existing phacoemulsification apparatus is that the needle ends in a ring-shaped end that is not a very effective emitter of ultrasonic sound waves and therefore the apparatus ultrasonic waves of relatively large amplitudes.

### SUMMARY OF THE INVENTION

The present invention provides an apparatus that overcomes or at least alleviates the disadvantages discussed above. An apparatus for removing the natural or crystalline lens (usually with cataracts) from a patient's eye includes a hand-held body with horn-shaped portion terminating in a needle. The horn-shaped portion provides mechanical energy for breaking up the natural lens.

An appropriate irrigating fluid (typically a salient aqueous solution) is provided through the handle and exits through one or more lateral opening in the tip into the anterior portion of the eye usually through a silicone sleeve surrounding the actual needle used to apply sonic waves for performing phacoemulisification of the lens. The lens detritus resulting from the emulsification process is aspirated through a central orifice in the needle tip. The tip is fabricated of a metallic material (titanium is customary but could be other suitable metal). A transducer acts as a sound generator and generates ultrasonic or sub-sonic sound waves that drive and vibrate the tip of the needle.

In one embodiment, the needle is formed of a plurality of prongs arranged in a circumferential symmetrically or asymmetrically configuration defining the tip of the needle about an aspiration orifice.

As previously mentioned, the hand piece is coupled to a suitable vibrating mechanism that vibrates the tip of the needle. The conventional practice until now has been to apply sound waves at an ultra-sonic range (typically 30-60 KHz) and normally do not contact the natural lens.

However the present inventor has found that, alternatively, the needle can be driven within the normal sonic range (typically 40-400 Hz). In this embodiment, the prongs preferably contact the lens nucleus and epinucleus and their vibration through both mechanical means and ultrasonic cavitation causes the lens to break up and form an emulsate.

The needle (typically made of titanium or similar materials) is attached to the horn and may have between two and five (or more) prongs that, in one embodiment, may be bent to as much 15-20 degrees toward the center of needle and its orifice. The prongs may be rounded at their ends to provide a potentially salutary effect to the capsule if it should be inadvertently engaged.

Depending on the configuration selected, the apparatus provides a number of advantages to the present state of the art:
1. The low frequency embodiment requires no coolant since no heat is generated. In the high frequency embodiment, less coolant maybe required.
2. Visibility using a multiple pronged-needle fragmenting system may be enhanced making the risk of misjudging emulsification depth less likely.
3. An apparatus with a multi- pronged tip uses the cumulative effect of the energy delivered through the prongs to the fragmentation process; in association with the re-directed fluidics described herein which may make for an efficient and less chaotic process at the needle tip. The needle prongs may be angled to increase efficient cutting.
4. Tips may be energized to act in transverse, oscillatory longitudinal or rotational modes.
5. The lateral flow of the irrigating fluid from the needle results in a more efficient procedure with less repulsion of lens material away from the cutting process and towards the posterior section of the eye

### BRIEF DECRIPTION OF THE DRAWINGS

FIG. 1A shows a block diagram of an apparatus constructed in accordance with this invention;

FIG. 1 shows an enlarged side orthogonal sectional view of the needle tip for one embodiment of the apparatus of FIG. 1A;

FIG. 2 shows an enlarged orthogonal section of an alternate embodiment of the invention;

FIG. 2A shows a side sectional view of the embodiment of FIG. 2;

FIGS. 3A, 3B and 3C show various alternate configurations for the needle of FIG. 1 and its prongs; and

FIG. 4 shows an orthogonal view of an irrigation aperture with a flap constructed in accordance with this invention;

FIG. 5 shows an orthogonal view of another embodiment of the invention.

### DETAILED DESCRIPTION OF THE PREFERRED EMBODIMENT

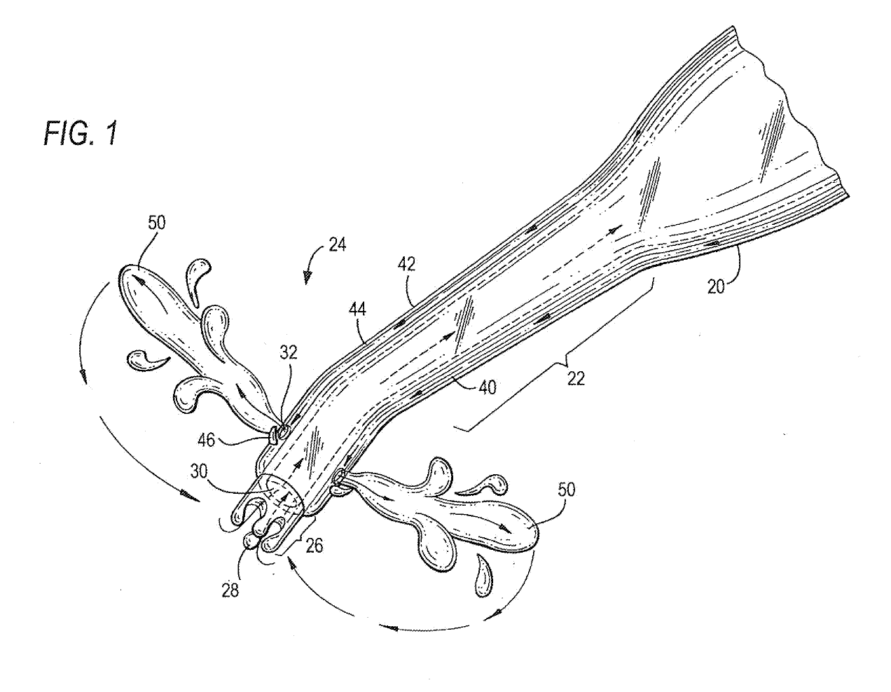

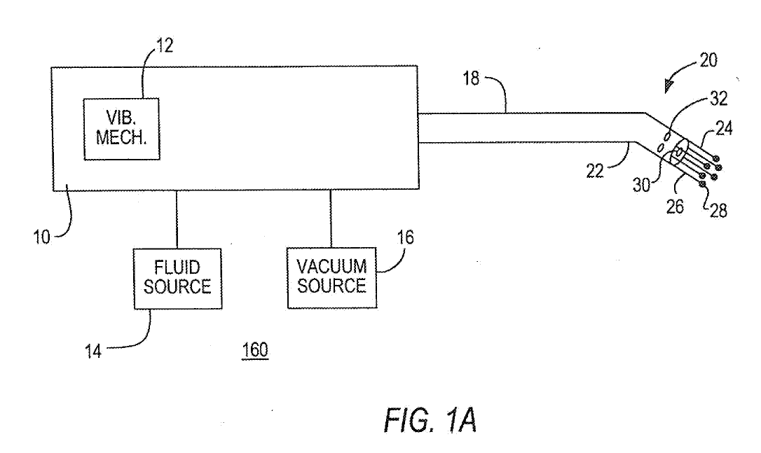

Referring first to FIG. 1A, an apparatus 100 constructed in accordance with this invention includes a handle 10 that includes a vibrating mechanism 12 and is connected to a fluid source 14 that provides irrigating fluid and a vacuum source 16. One end of the handle 10 is provided with a horn 18 terminating with a needle 20. The needle 20 is preferably bent, as at 22. The needle 20 includes a tip 24. In one embodiment, the tip 24 is formed of a plurality of prongs 26 extending generally coaxially along needle 20. The prongs 26 are terminated in one embodiment with crowns 28. The prongs 26 are disposed circumferentially around a central aspiration aperture 30. Tip 24 further includes a plurality of irrigation apertures 32.

The vibrating mechanism 12 may be, for example, a transducer that provides excitation for the mechanical vibration of the tip 24 (at either a sonic, e.g. 40-400 Hz or ultrasonic, e.g. 30-60 KHz, frequency range) to cause the natural lens in the capsular bag of an eye (not shown) to break up, as discussed in more detail below. This vibration is transmitted to prongs (described in more detail below) through a metal tube and these elements cooperate to cause the prongs to move in at least one of a translational motion, rotational motion, etc.

The horn 18 is typically a housing incorporating an integrated metal tube which tapers to fit the casing as it approaches the cut-outs that represent the emulsifying needle prongs. As is known in the art, in one embodiment, the needle prongs are attached to the horn assembly in a manner that allows them to be selectively detached and disposed after use. In other words, the needle and its prongs may be disposable. In one iteration the needle prongs could be sectioned elements of the integrated titanium tube attached to the horn or independent metallic materials designed for this purpose.

As shown in FIG. 1, the tip 24 includes a central tube 40 (typically made of titanium) preferably made of a metallic or other similar relatively stiff material. The tube 40 is surrounded by a sleeve 42. The sleeve is often manufactured of silicone but may be of other materials, and is provided with either an annular cannula 44 or one or more tubular longitudinal openings extending from the handle to the irrigation apertures 32. The sleeve is attached tightly around the central tube 40 past the irrigation apertures 32.

As mentioned above, preferably the tip 24 is formed of a plurality of prongs 26 having crowns 28. The vibrating mechanism 12 and tube 40 cooperate to cause the prongs 26 to vibrate in one of a series of controlled motions. The optimal efficiency mode of vibration of these prongs is dependent on the length, thickness and material of the prongs, the size and weight of the crowns 28 and the angle of the prongs 26 with respect to the longitudinal axis of the tube 40. The multiple pronged tip is configured and arranged to increase the efficiency of emulsification (as compared to previous devices) through contact to lens material.

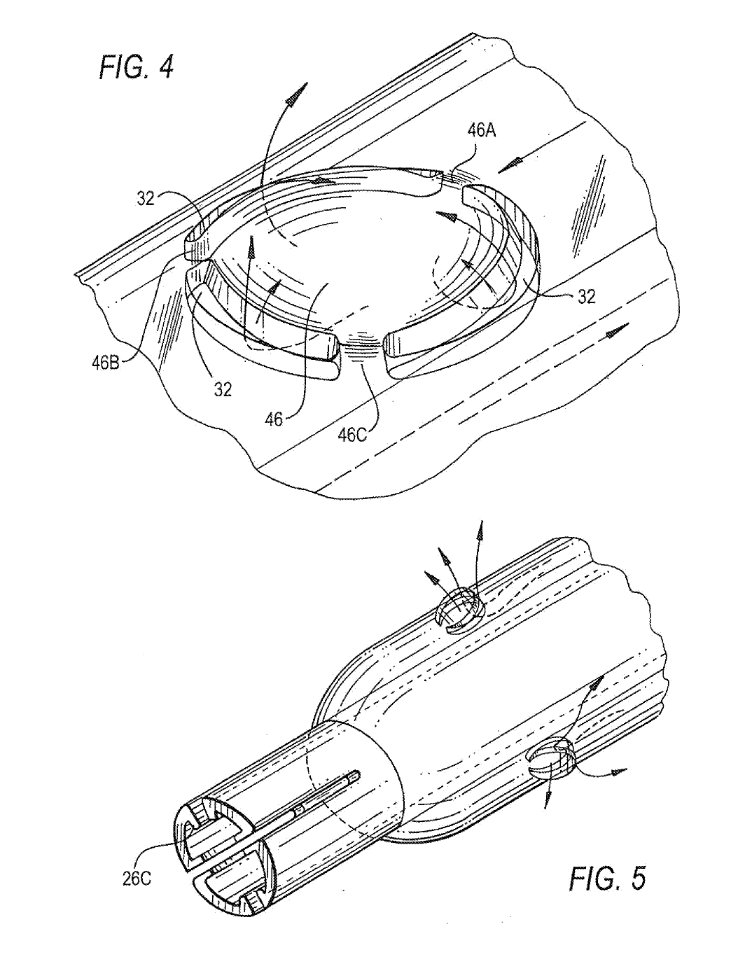

A typical conventional phacoemulsification device includes a handle similar top handle 10 in FIG. 1A attached to a horn terminating with a needle having straight tip as opposed to the prongs discussed above. In such a device, a sleeve, usually made of silicone, extends along the horn and is adapted to conduct fluid directed at the tip for cooling the tip and as the irrigation fluid. In one aspect of the present invention, a phacoemulsificiation apparatus having a similar structure is provided, however, the conventional sleeve is replaced with a sleeve having the baffles shown in FIGS. 1 and 4 directing fluid away rather than in proximity of the tip. The circumferential end portion of the sleeve near or adjacent to the tip hugs the tube 40 and forms a seal to prevent fluid from reaching the tip.

The apparatus is used as follows. A small opening is first made in the capsular bag of the eye. The lens is either engaged within the capsular bag or the lens is dislocated anteriorly. Either way in the next steps, the tip 24 of the needle 20 is made to have contact with the nucleus of the lens. This step is facilitated by the bent 22 formed in the needle.

Next, the vibrating mechanism is started coincidentally with the infusion of irrigating fluid 50 which is introduced through the cannula 44.

Preferably the irrigation apertures 32 are covered or closed by flexible baffles or other somewhat flexible members arranged and constructed to redirect fluid to a more lateral direction 46. The sleeve 42 (made, for example, from silicone) presents a substantially continuous outer surface as the needle 20 is juxtaposed or in contact to the lens nucleus. However, once the tip 24 has engaged the nuclear lens material either outside or within the capsular bag, irrigation fluid (usually under pressure) is provided from source fluid 14 through the cannula 44. The fluid pushes the baffles 46 open and then exits into the eye forming a plume 50 that extends at an angle away from the prongs 26. Preferably, the fluid forms a plume having an axis forming an angle of approximately 90 degrees with the longitudinal axis of tube 40. Of course, this angle may also be more or less than 90 degrees. As the prongs 26 vibrate, the natural or crystalline lens of the eye is broken up and emulsified.

The central aperture 30 is connected through central tube 40 to the vacuum source 16 causing fluid and emulsate to flow through the central aperture 30 and out the eye to the machine console. Using the invention and its redirected infusion apertures 32, the lens nuclear fragments are readily emulsified by the vibrating prongs 26 and detritus is more efficiently removed from the eye and is less likely to be lost to aspiration and left in the eye.

In prior art devices, irrigation fluid exits between or close to the prongs (for cooling the prongs) and is directed axially along the prongs forming a fluid flow in direction X in FIG. 1. Detritus formed at or by the prongs is caught up in this flow and is carried away from the tip into remote zones often beyond the capsular bag and to other parts of the eye. As a result of the inefficiencies of prior art emulsification of nuclear lens may take longer, and in some cases the removal may be incomplete, especially when the detritus reaches other parts of the eye. In the present invention, instead a toroidal flow Y is established that is salutary to the aspiration functions of the device and since it is less repulsive to fragmenting lens material will allow for greater efficiency of ultrasonic or subsonic emulsification. Therefore detritus is more directly aspirated towards the aperture 30 and not towards remote areas of the eye. As a result, the detritus is removed more efficiently and/or faster than in prior art devices.

For the low frequency embodiment, the configurations shown are even more advantageous because fluid is not required to cool the prongs, since at such frequencies, and without significant cavitation, damaging heat is not produced.

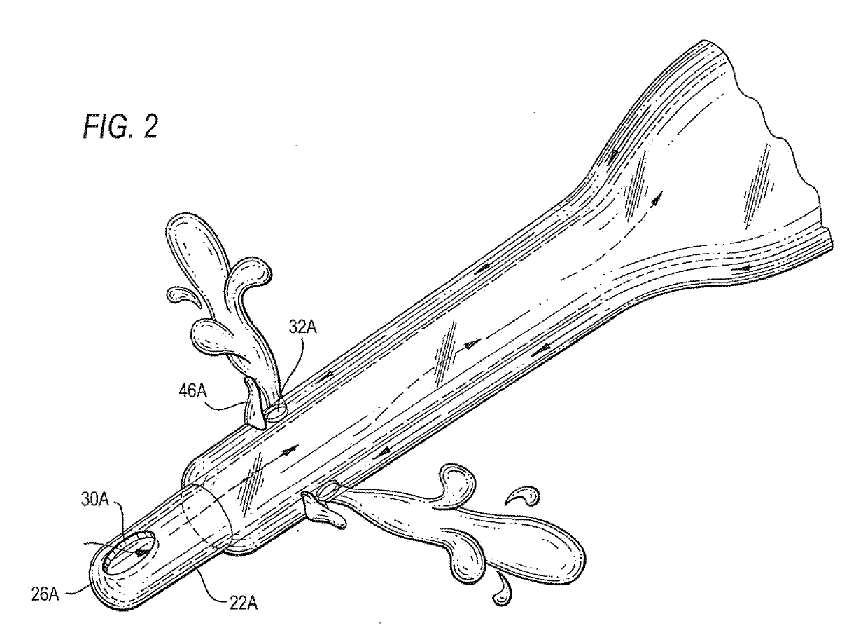

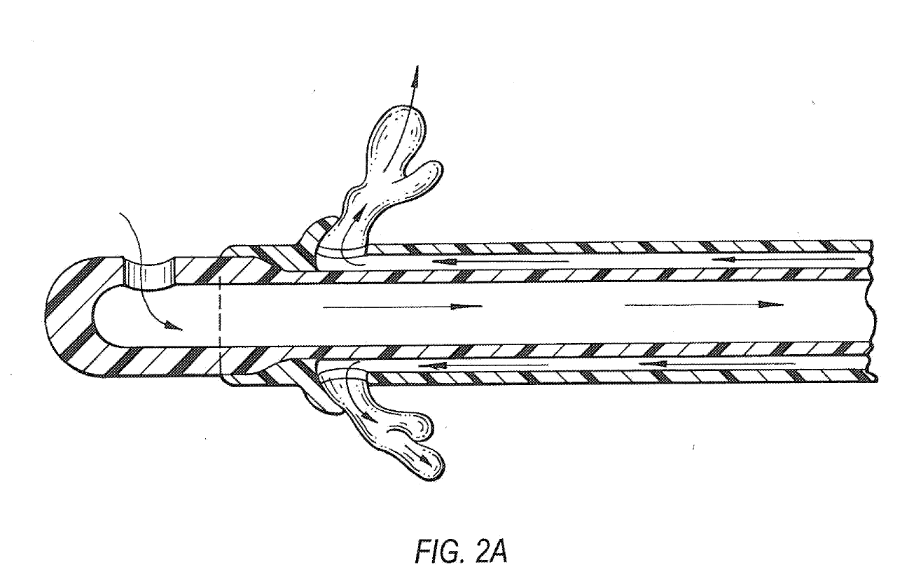

In one embodiment shown in FIG. 2, the tip 24A is somewhat bullet shaped with a round nose 26A rather than several prongs. Excitation for breaking up the lens is provided at the nose 26A. In this embodiment, irrigation fluid is still provided through several apertures 32A (with flaps 46A) at a position axially recessed from the tip 22A. The fluid then picks up the detritus and is vacuum out from the capsular bag through the aperture 30A.

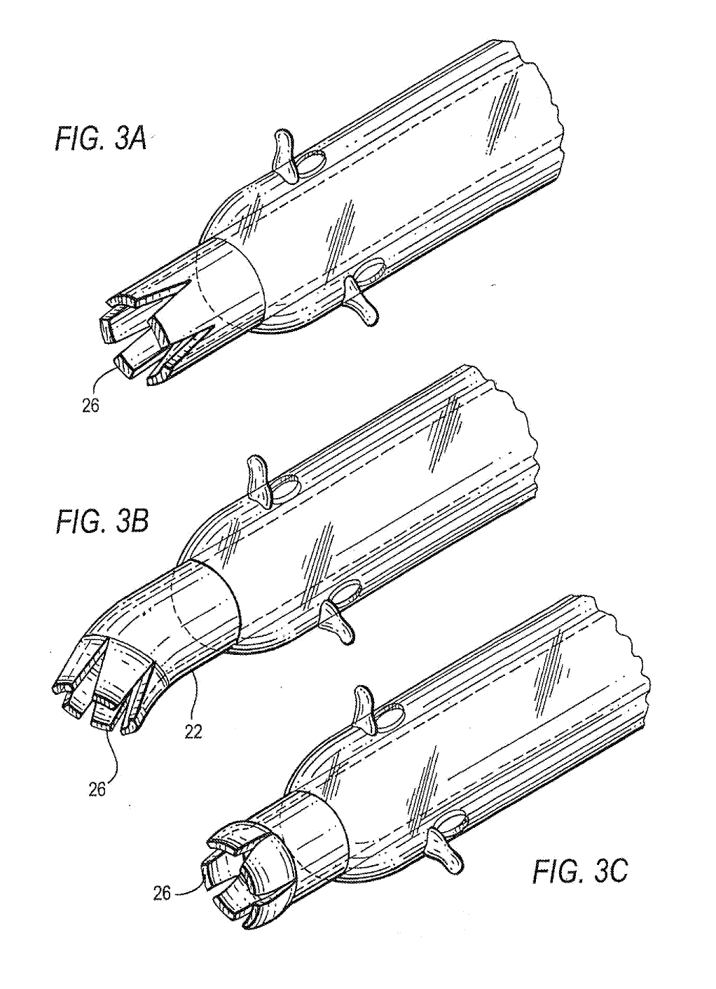

The prongs and the needle 20 can be arranged into several configurations. In FIG. 1 the needle is provided with bend 20 and the prongs 26 are disposed generally axially. In other embodiments, the prongs may be angled (for example, by 10-20 degrees) toward the axis of the needle thereby increasing their effectiveness. This angulation is balanced to the need for efficient aspiration versus requirements for cutting. FIG. 3A shows an embodiment in which needle 20 and the prongs 26 extend coaxially with no bend in the needle or the prongs. In the embodiment of FIG. 3B, the needle includes bend 22 and the prongs 26 are angled radially inwardly. In FIG. 3C the prongs 26 are angled radially inwardly as discussed above, but the needle has no band.

The multiplicity of needle prongs may have various degrees of arc and length to the longitudinal perspective from the hand-piece. As cut from a tubular device the needle prongs, as described, would be partial elements of the classic circumferential phacoemulsification needle (consider a half pipe as the minimal design resulting in two needle prongs). Additionally the needle-prongs could be bent to varying degrees according to the inherent power described by that advantage.

The following are approximate dimensions of the various elements discussed:

Needle 18 may have a circular or ovoid cross-section at its tip 24 would vary from 0.8 mm to 1.5 mm.

The ID of tube 40 is approximately 0.5 to 0.9 mm.

The aperture 30 has a diameter of about 0.65 mm to 1.4 mm.

The OD of the sleeve 42 is in the range of 1.4 mm to 1.8 mm.

In a flared tip design the OD of a circle defined by the prongs 26 is approximately 0.95 mm.

The prongs 26 would vary from approximately 0.2 mm to 1.0 mm in length.

The plum formed by the irrigation fluid as it exits from the irrigation apertures is disposed at an angle of at least 90 degrees with the axis of the tube 40, and preferably greater than 90 degrees.

The silicone sleeve is drawn down along the shaft of the hand-piece stopping with a tight seal above the needle prongs and positioned in such a way as to provide the most efficient maintenance of the anterior chamber without setting up undue turbulence in relation to nuclear lens material at the lumen of the needle prong arrangement.

In a preferred embodiment, the irrigation apertures 32 through which fluid is expelled into the anterior chamber are provided with deflecting, collapsible flaps acting as the baffles 46 set along the silicone sleeve as shown in FIG. 4. Each flap includes a central portion 46C connected at one point with a hinge 46B to an edge of irrigating aperture 32, and one or more leashes 46A that are either very flexible and expand when fluid pressure is applied to the portion 46C to allow the portion 46C to separate from the aperture 32, or are connected only to main portion 46C and are provided to position the main portion 46C properly within the aperture 32. In this latter configuration, the central portion is biased toward the aperture 32 by the hinge 46B.

When infusion fluid is directed down the sleeve 42 surrounding the tube 40, the flaps 46 are made to inflate outward or otherwise open as a clam-like design while still partially fixed by hinge 46B. Further the flaps may be partially leashed proximally to the proximal edges of the port at the sleeve (more than one leash may be considered depending on the port size) in order to limit the excursion of the flap. Importantly, when no infusion fluid is provided, the flaps are folded along the sleeve 42 to act as a ramp to smooth insertion or removal of the instrument through the corneal or scleral wound. When fluid is not actively flowing in a vigorous manner, the flap will be collapsed or partially collapsed facilitating removal of the hand-piece from the eye. Aiding in the directing infusion flow a circumferential hub of thickened silicone just at the margin of distal port position would act to abruptly redirect fluid flow towards the ports.

In one embodiment, foot-pedal (not shown) coupled to the hand piece 10, can be placed in one of several positions (a standard arrangements for a generic phacoemulsification device) fluid flowing is initiated with some degree of force opens the flap to a prescribed degree allowing deflected fluid to flow across the capsular bag relatively lateral to the port.

The flap or baffle may have a central portion that is round, ovoid or some other distinguishable shape of silicone or some other flexible material continuous at both the hinge and leash across the distal and proximal edges of the edges of the irrigation apertures respectively which may be round or oval (or variously shaped) along the silicone sleeve just proximal to the metallic phacoemulsification tip 24. The 42 sleeve is tightly fit at its distal end, preventing or limiting fluid flow directly across the tip which would otherwise be directed into the posterior chamber.

The outer diameters of the irrigation apertures may be variously sized (e.g. 1.5-2 mm) In association with the intended rate of flow into the chambers of the eye.

As previously mentioned, the tongs 26 can be created from a tube by making longitudinal cuts. The corners of the tongs can be rounded as illustrated in FIG. 5.

Obviously numerous modifications may be made to this invention without departing from its scope as defined in the appended claims.

I claim:

1. An apparatus for performing surgery in the anterior, posterior chamber capsular of the eye and within the bag of an eye comprising:
a vibration mechanism; and
a handle connected to a source of irrigation fluid, and a needle having a tip having a member coupled to said vibration mechanism and adapted to vibrate, said tip including an irrigation aperture receiving irrigation fluid, said irrigation aperture being arranged to expel said irrigation fluid from said needle away from said tip.

2. The apparatus of claim 1 wherein said irrigation aperture is spaced longitudinally away from said member.

3. The apparatus of claim 1 wherein said aperture is configured to generate said plume at an angle of at least 90 degrees with respect to a longitudinal axis of said needle.

4. The apparatus of claim 3 wherein said angle is over 90 degrees,

5. The apparatus of claim 1 wherein said tip includes an aspiration aperture disposed at said tip and near said member, said irrigation aperture and said aspiration aperture cooperating to generate an irrigation plume between said irrigation and said aspiration aperture and away from said member.

6. The apparatus of claim 5 wherein said irrigation plume is toroidal.

7. The apparatus of claim 1 wherein said member includes a plurality of prongs.

8. A device associated with an apparatus with a vibrating mechanism for providing surgery in the capsular bag of an eye, said apparatus comprising:
an elongated cylindrical tube terminating at one end with a tip and being adapted to couple to said apparatus at said other end; and
a tip formed on said cylindrical tube having a member coupled to the vibration mechanism and adapted to vibrate, said tip including an irrigation aperture receiving irrigation fluid, said irrigation aperture being arranged to expel said irrigation fluid from said needle away from said tip.

9. The device of claim 8 wherein said irrigation aperture is spaced longitudinally away from said member.

10. The device of claim 8 wherein said aperture is configured to generate said plume at an angle of at least 90 degrees with respect to a longitudinal axis of said needle.

11. The device of claim 10 wherein said angle is over 90 degrees,

12. The device of claim 8 wherein said tip includes an aspiration aperture disposed at said tip and near said member, said irrigation aperture and said aspiration aperture cooperating to generate an irrigation plume between said irrigation and said aspiration aperture and away from said member.

13. The device of claim 12 wherein said irrigation plume is toroidal.

14. The device of claim 8 wherein said member includes a plurality of prongs.

15. The device of claim 8 wherein said member is vibrating in a sonic frequency range.

16. The device of claim 8 wherein said member is vibrating at a supersonic frequency range.

17. The device of claim 8 further comprising a flap sized and shaped to fit over said irrigating aperture.

18. The device of claim 17 wherein said flap is arranged to shape said plume.

19. A method of performing phacoemulsification of a lens in an eye using a needle having a tip with a vibrating member arranged and constructed to apply vibrating said lens at a predetermined frequency to shatter or emulsify said lens, said tip including an irrigating aperture and an aspirating aperture, said method comprising the steps of:
applying energy to said vibrating member to cause said vibration; and
generating a plume of irrigating liquid from said irrigating aperture toward said aspirating aperture, said plume being shaped and sized to receive and aspirate pieces of said lens, said plume being shaped to extend around said member.

20. The method of claim 19 wherein said step of shaping including shaping said plume into a toroidal form.
